SEQUENCE LISTING (4 sequences)

SEQ ID NO: 1 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 2 - DNA, 38 bp, Unknown
  oligonucleotide primer
ccccccccgctagcgaagcccctcctcggcgtcctggt

SEQ ID NO: 3 - DNA, 40 bp, Unknown
  oligonucleotide primer
ccccccccaccggtccgcccaggctcccaggaaggagaaa

SEQ ID NO: 4 - DNA, 42 bp, Unknown
  oligonucleotide primer
ccccccccgaattcttacctcatccttctccacaacagagct

(19) United States
(12) Patent Application Publication
(10) Pub. No.: US 2010/0266530 A1
(43) Pub. Date: Oct. 21, 2010

(54) FcRN ANTIBODIES AND USES THEREOF

(75) Inventors: Derry Charles Roopenian, Salisbury Cove, ME (US); Shreeram Akilesh, St. Louis, MO (US); Gregory James Christianson, Seal Cove, ME (US); Stefka Petkova, Ellsworth, ME (US); Petko M. Petkov, Ellsworth, ME (US); Thomas J. Sproule, Trenton, ME (US); Emanuele Pesavento, Creazzo (IT)

Correspondence Address: ROPES & GRAY LLP, PATENT DOCKETING 39/41, ONE INTERNATIONAL PLACE, BOSTON, MA, 02110-2624, US

(73) Assignee: THE JACKSON LABORATORY, Bar Harbor, ME (US)

(21) Appl. No.: 11/919,596

(22) PCT Filed: Apr. 14, 2006
(86) PCT No.: PCT/US2006/014182

§ 371 Date: Mar. 19, 2010

Related U.S. Application Data
(60) Provisional application No. 60/676,412, filed on Apr. 29, 2005.

(51) Int. Cl. A61K 39/395; C07K 16/28; C12N 5/07; A61K 38/21; A61K 38/20; C12N 15/13; G01N 33/53; A61K 49/00; A61P 37/04; A61P 37/06

(52) U.S. Cl. 424/85.2; 530/389.1; 530/388.22; 530/387.3; 530/391.1; 530/391.3; 435/334; 424/172.1; 424/85.7; 424/85.5; 536/23.53; 436/501; 800/3

(57) ABSTRACT

In certain embodiments, this present invention provides polypeptide compositions (e.g., antibodies and antigen binding portions thereof that bind to FcRn), and methods for modulating FcRn activity. In other embodiments, the present invention provides methods and compositions for treating autoimmune disorders.

### RELATED APPLICATIONS

This application claims priority to U.S. Provisional Application No. 60/676,412, filed Apr. 29, 2005, the specification of which is hereby incorporated herein by reference in its entirety.

### FUNDING

Work described herein was funded, in whole or in part, by National Institutes of Health Grant Number NIH DK57597. The United States government has certain rights in the invention.

### BACKGROUND OF THE INVENTION

Antibodies have been known since before the 20th century to play an important role in immunological protection against infectious organisms. The immune system cells that produce antibodies are B-lymphocytes. There are four major classes: immunoglobulin M (IgM), IgG, IgA, and IgE, but IgG is by far the most prevalent class, comprising about 90% of all antibodies in adults. Each class of antibody has a specific role in immunity, including primary and secondary immune responses, antigen inactivation and allergic reactions. IgG is the only class of antibody that can pass the placental barrier, thus providing protection from pathogens before the newborn's immune system develops. Antibody molecules have two ends. One end is the antigen-specific receptor, which is highly variable and engenders each antibody with the capacity to bind a specific molecular shape. The other end, referred to as Fc, has sequence and structural similarities within a class and confers the ability to bind to receptors on immune cells. In a perfectly operating immune system, the diverse specificities of the antigen specific receptor engenders the host with a diverse repertoire of antibodies with the ability to bind to a wide array of foreign infectious microorganisms, the result being destruction of the microbe and immunity.

Autoimmune diseases occur when the immune system erroneously senses that normal tissue is foreign and attacks it. One of the most prevalent immunological participants in autoimmune destruction are auto-antibodies, which are normal antibody molecules that have gone awry and destroy normal tissue. This leads to many types of autoimmune diseases, including systemic lupus erythematosus (SLE). SLE is a prototypic disease of systemic antibody dysregulation with the common feature of hypergammaglobulinemia, anti-DNA features and anti-nuclear protein antibodies, and immune complexes that accumulate at many sites including the kidney glomeruli, vascular system, joints and skin (Theofilopolos and Dixon, 1985, Adv. Immunol. 37: 296-390; Theofilopolos and Dixon, 1981, Immunol. Rev., 1981, 55:179-215; Boumpas et al., 1995, Ann. Int. Med. 122:940). The severity can range from mild to very severe, from minimally debilitating to lethal. There are currently few effective treatments for autoimmune diseases.

### SUMMARY OF THE INVENTION

Accordingly, it is the goals of the application to develop more effective compositions and methods for manipulating antibody concentrations as a way to treat autoimmune diseases.

In certain embodiments, the present invention provides an isolated antibody or antigen binding portion thereof that binds to an epitope on human FcRn. The isolated antibody is referred to herein as an FcRn antibody. The FcRn antibody or the antigen-binding portion thereof binds epitopes of human FcRn and selectively inhibits the binding of the Fc portion of IgG antibody to human FcRn, but does not inhibit the binding of human albumin to human FcRn. In certain specific embodiments, the FcRn antibody is a monoclonal antibody. In certain embodiments, the FcRn antibody or antigen binding portion thereof can be administered to a human. In certain cases, the FcRn antibody or antigen binding portion thereof selectively decreases the serum half-life of a human IgG but does not substantially decrease the serum half-life of human albumin in vivo. In other cases, the FcRn antibody or antigen binding portion thereof ameliorates or inhibits inflammatory lesions induced by a human autoantibody in a person. Examples of the antibody include, but are not limited to, FcRn antibodies denoted herein as DVN21 and DVN24. The subject FcRn antibody includes, but is not limited to, a recombinant antibody, a humanized antibody, a chimeric antibody, a human antibody, a bispecific or multispecific antibody, and an isolated antigen-binding portion (e.g., an Fab fragment, an F(ab′)2 fragment, and an Fv fragment CDR3).

In certain embodiments, the FcRn antibody or antigen-binding portion thereof is selected for its ability to bind live cells expressing FcRn (e.g., a labeled FcRn protein). In certain cases, the antibody or antigen-binding portion thereof is selected in vivo for its ability to decrease the serum half-life of a human IgG and inability to substantially decrease the serum half-life of human albumin. In other cases, the antibody or antigen-binding portion thereof is selected in a transgenic mouse which is deficient in the endogenous FcRn gene but has a transgene encoding human FcRn.

In further embodiments, the isolated FcRn antibody or antigen binding portion thereof is covalently linked to an additional functional moiety, such as a label. In specific embodiments, the label is suitable for detection by a method selected from the group consisting of fluorescence detection methods, positron emission tomography detection methods and nuclear magnetic resonance detection methods. For example, the label is selected from a fluorescent label, a radioactive label, and a label having a distinctive nuclear magnetic resonance signature. In certain cases, the additional functional moiety confers increased serum half-life on the antibody or antigen binding portion thereof. To illustrate, the additional functional moiety comprises a polyethylene glycol (PEG) moiety or a biotin moiety.

In certain embodiments, the present invention provides a hybridoma cell line that produces an FcRn antibody as described above. In certain embodiments, the hybridoma cell line produces a monoclonal FcRn antibody that selectively inhibits the binding of the Fc portion of IgG antibody to human FcRn, but does not inhibit the binding of human albumin to human FcRn. For example, the hybridoma cell line produces an FcRn antibody such as DVN21 and DVN24.

In certain embodiments, the present invention provides a composition comprising at least one FcRn antibody or antigen-binding portion thereof as described above and a pharmaceutically acceptable carrier, excipient, or stabilizer. The composition can further comprise an immunostimulatory agent, an immunomodulator, or a combination thereof. For example, the composition comprises an immunomodulator, such as but not limited to, alpha-interferon, gamma-interferon, tumor necrosis factor-alpha, or a combination thereof. As another example, the composition comprises an immunostimulatory agent including but not limited to, interleukin-2, immunostimulatory oligonucleotides, or a combination thereof.

In certain embodiments, the present invention provides an isolated nucleic acid molecule encoding a FcRn antibody or antigen-binding portion thereof as described above.

In certain embodiments, the present invention provides a method for inhibiting FcRn mediated IgG protection in an individual. Such method comprises administering to an individual in need of inhibition of FcRn mediated IgG protection an FcRn antibody in sufficient amounts to selectively inhibit binding of human FcRn to a human IgG but not to human albumin. For example, such an FcRn antibody can be administered to an individual with an autoimmune disease. Examples of autoimmune diseases include, but are not limited to, SLE, insulin resistant diabetes, myasthenia gravis, polyarteritis, autoimmune thrombocytopenic purpura, cutaneous vasculitis, bullous pemphigoid, pemphigus vulgaris, pemphigus foliaceus, Goodpasture's syndrome, rheumatoid arthritis, Kawasaki's disease, and Sjogren's syndrome.

In certain embodiments, the present invention provides a method of preventing or treating an autoimmune disease in a patient. Such method comprises administering an FcRn antibody to a patient in sufficient amounts to prevent or treat the autoimmune disease. In a specific embodiment, the FcRn antibody administered selectively inhibits binding of the Fc portion of IgG antibody to human FcRn, but does not inhibit binding of human albumin to human FcRn. Examples of the autoimmune diseases include, but are not limited to, SLE, insulin resistant diabetes, myasthenia gravis, polyarteritis, autoimmune thrombocytopenic purpura, cutaneous vasculitis, bullous pemphigoid, pemphigus vulgaris, pemphigus foliaceus, Goodpasture's syndrome, rheumatoid arthritis, Kawasaki's disease, and Sjogren's syndrome. The isolated antibody or antigen binding portion thereof can be administered to an individual systemically or locally. In certain cases, the method further comprises administering to a patient an immunomodulator such as alpha-interferon, gamma-interferon, tumor necrosis factor-alpha, or a combination thereof.

In certain embodiments, the present invention provides an in vitro method of identifying an inhibitor that selectively inhibits binding of human FcRn to a human IgG but not to human albumin (a “selective FcRn inhibitor”). Such method comprises: (a) contacting a candidate inhibitor with human FcRn, a human IgG, and human albumin; (b) assaying for binding of human FcRn to the human IgG in the presence of the candidate inhibitor, as compared to binding of human FcRn to the human IgG in the absence of candidate inhibitor; and (c) assaying for binding of human FcRn to human albumin in the presence of the candidate inhibitor, as compared to binding of human FcRn to human albumin in the absence of candidate inhibitor. The desired selective FcRn inhibitor inhibits binding of human FcRn to the human IgG but not to human albumin. In certain embodiments, the selective FcRn inhibitor is selected from an antibody, a polypeptide, a synthetic peptide, a peptidomimetic, or a small molecule. In certain cases, the selective FcRn inhibitor is either a fusion protein comprising an Fc portion of an IgG polypeptide. Alternatively, the selective FcRn inhibitor is an Fc portion of an IgG polypeptide.

In further embodiments, the present invention provides alternative in vitro method of identifying an inhibitor that selectively inhibits binding of human FcRn to a human IgG but not to human albumin. Such method comprises: (a) contacting a candidate inhibitor with human FcRn and a human IgG under conditions appropriate for binding of the human FcRn to the human IgG; (b) assaying for binding of human FcRn to the human IgG in the presence of the candidate inhibitor, as compared to binding of human FcRn to the human IgG in the absence of candidate inhibitor; (c) contacting a candidate inhibitor to human FcRn and human albumin under conditions appropriate for binding of the human FcRn to human albumin; and (d) assaying for binding of human FcRn to human albumin in the presence of the candidate inhibitor, as compared to binding of human FcRn to human albumin in the absence of candidate inhibitor. The desired selective FcRn inhibitor inhibits binding of human FcRn to the human IgG but not to human albumin. In certain embodiments, the selective FcRn inhibitor is selected from an antibody, a polypeptide, a synthetic peptide, a peptidomimetic, or a small molecule. In certain cases, the selective FcRn inhibitor is a fusion protein comprising an Fc portion of an IgG polypeptide. Alternatively, the selective FcRn inhibitor is an Fc portion of an IgG polypeptide.

In certain embodiments, the present invention provides an in vivo method of identifying an agent that selectively reduces the half-life of human IgG but not the half-life of human albumin. Such method comprises: (a) administering a candidate agent and a tracer human IgG to an FcRn^{-/-}/huFcRn⁺ transgenic mouse; (b) determining the half-life of the tracer human IgG in the mouse in the presence of the candidate agent, as compared to the half-life of the tracer human IgG in the absence of candidate agent; (c) administering the candidate agent and a tracer human albumin to the FcRn^{-/-}/huFcRn⁺ transgenic mouse; and (d) determining the half-life of the tracer human albumin in the mouse in the presence of the candidate agent, as compared to the half-life of the tracer human albumin in the absence of candidate agent. If the candidate agent reduces the half-life of the tracer human IgG but not the half-life of the tracer human albumin, the candidate agent is an agent that selectively reduces the half-life of human IgG but not the half-life of human albumin. The agent can be, for example, selected from an antibody, a polypeptide, a synthetic peptide, a peptidomimetic, and a small molecule. In certain cases, the agent is a fusion protein comprising an Fc portion of an IgG polypeptide. Alternatively, the agent is an Fc portion of an IgG polypeptide.

In certain embodiments, the present invention is an in vivo method of identifying an agent that selectively reduces the half-life of human IgG but not the half-life of human albumin. Such method comprises: (a) administering a candidate agent, a tracer human IgG, and a tracer human albumin to an FcRn^{-/-}/huFcRn⁺ transgenic mouse; (b) determining the half-life of the tracer human IgG in the mouse in the presence of the candidate agent, as compared to the half-life of the tracer human IgG in the absence of candidate agent; and (c) determining the half-life of the tracer human albumin in the mouse in the presence of the candidate agent, as compared to the half-life of the tracer human albumin in the absence of candidate agent. A desired agent selectively reduces the half-life of the tracer human IgG but not the half-life of the tracer human albumin. The agent can be, for example, selected from an antibody, a polypeptide, a synthetic peptide, a peptidomimetic, or a small molecule. In certain cases, the agent is a fusion protein comprising an Fc portion of an IgG polypeptide. Alternatively, the agent is an Fc portion of an IgG polypeptide.

In certain embodiments, the present invention provides use of an isolated FcRn antibody or antigen binding portion thereof to make a pharmaceutical preparation for treating an autoimmune disease. In certain cases, the FcRn antibody selectively inhibits the binding of human FcRn to the Fc portion of IgG antibody, but not to human albumin. In a specific embodiment, the FcRn antibody is a monoclonal antibody.

In certain embodiments, the present invention provides use of an isolated FcRn antibody or antigen binding portion thereof to promote clearance of radioactive antibodies or antibody conjugated toxins. In certain embodiments, these radioactive antibodies or antibody conjugated toxins are used for imaging or treatment of cancer. In a specific embodiment, the FcRn antibody is a monoclonal antibody.

### BRIEF DESCRIPTION OF THE DRAWINGS

FIGS. 1A-1B show construction and validation of hFcRn constructs.

FIG. 1A: Schematic of hFcRn cDNA constructs. ssECTM (signal sequence-GFP-ectodomain-transmembrane domain), and ECTM (signal sequence-ectodomain-transmembrane domain). Cloning sites and FcRn codon positions are indicated. The STOP codon is denoted by *.

FIG. 1B: Flow cytometric analysis of pH-dependent binding of hIgG to ECTM and ssECTM transfected HEK 293 cells.

FIGS. 2A-2D show flow cytometry of albumin and hIgG binding to hFcRn.

FIG. 2A: Binding of HSA or IgG to ssECTM cells. (1 & 6) Ctrl HSA, biotinylated goat anti-HSA+SA-APC. (2 & 7) Ctrl hIgG, goat anti-hIgG-PE. (3 & 8) HSA binding, HSA+biotinylated goat anti-HSA+SA-APC. (4 & 10) HSA binding, human serum+biotinylated goat anti-HSA+SA-APC. (5 & 9) hIgG binding, human serum+goat anti-hIgG-PE.

FIG. 2B: Binding of HSA-biotin to ssECTM cells. (1 & 3) Ctrl HSA, SA-APC. (2 & 4) HSA binding, biotinylated-HSA+SA-APC.

FIGS. 2C and 2D: Binding of HSA-biotin and hIgG₃-AF₆₄₇ to (C) ECTM and (D) HEK293 cells. Ctrl hIgG₃, no treatment; hIgG₃ binding, hIgG₃-AF₆₄₇; Ctrl HSAbio, SA-APC; HSAbio binding, HSA-biotin+SA-APC. For an internal negative control, a population of GFP-hFcRn negative cells was deliberately maintained with the GFP-hFcRn positive ssECTM and ECTM cells. Relative mean fluorescence intensity (MFI) is the ratio between MFI of the hFcRn-GFP positive population and MFI of hFcRn-GFP negative population. The bar graphs are the mean±s.e.m. (see M&M) of at least 4 independent experiments.

FIGS. 3A-3B are graphs showing results of competition between HSA and hIgG for binding hFcRn. ssECTM cells were incubated with the indicated doses of unlabeled hIgG (triangle), HSA (square), or hTF (circle), and then either hIgG-AF₆₄₇ or HSA-biotin was added. Assays were performed at pH 6.

FIG. 3A: Competition vs. 50 μg/ml hIgG-AF₆₄₇.

FIG. 3B: Competition vs. 250 μg/ml HSA-biotin. Data are expressed as MFI of GFP-positive gated cells. Representative data from one of two experiments with similar results is shown. HIgG-AF₆₄₇ binding to ssECTM cells at pH 7.5 without competitor resulted in an MFI of 6. HSA-biotin/SA-PE binding to ssECTM cells at pH 7.5 without competitor resulted in an MFI of 4.

FIG. 4 shows data on the binding activity of anti-hFcRn mAbs at pH 7.5 and 6.0, and the ability of DVN24 to block the binding of hIgG to hFcRn at pH 6.0. For direct binding data (left and middle scattergrams, 1 μg of the indicated mAbs were incubated with 10⁶ ssECTM cells for 30 min at 4° C. in the indicated pH buffer. The ssECTM cells were then washed 2×, and incubated with phycoerythrin conjugated goat anti-mouse IgG (Southern Biotech, Birmingham, Ala.), and then analyzed by flow cytometry. For inhibition of hIgG (right scattergrams), the mAbs were added in a concentration of 10 μg to 10⁶ ssECTM cells for 30 min. at 4° C. in pH 6.0 buffer, washed 2×, incubated with 1 μg AlexiFluor₆₄₇-conjugated hIgG3, washed 2× and analyzed by flow cytometry.

FIGS. 5A-5B are graphs of data that show that certain anti-hFcRn mAbs selectively block binding of hIgG or HSA to hFcRn at pH 6.0.

FIG. 5A: Blockade of hIgG. 10⁶ ssETCM cells were incubated at 4° C. with increasing concentrations of purified anti-hFcRn mAbs, washed 2×, and then incubated with 1 μg AlexaFluor₆₄₇-conjugated hIgG for 1 hour at 4° C. The ssECTM cells were then washed 2× and analyzed by flow cytometry.

FIG. 5B: Blockade of HSA. 10⁶ ssETCM cells were incubated at 4° C. with increasing concentrations of purified anti-hFcRn mAbs, washed 2×, and then incubated with 1 μg biotin-conjugated HSA. The ssECTM cells were then washed 2×, incubated with streptavidin-phycoerythrin, washed 2×, and analyzed by flow cytometry. All incubations were performed in pH 6.0 buffer. Data are presented as mean fluorescence intensity (MFI) of the GFP-positive gated cells.

FIG. 6 is a graph of data that show that administration of DVN24 mAbs reduces the serum concentration of hIgG. 100 μg of tracer hIgG was injected intraperitoneally into groups of 5 mouse FcRn−/− hFcRn Line 276 transgenic mice on day 0. Varying concentrations of DVN24 or 1000 μg of an isotype-matched negative control mAb was injected intraperitoneally on days 2, 3, and 4. Sera from serial eye bleeds were then analyzed by ELISA for the concentration of injected hIgG tracer. Data are presented based on the % of serum tracer hIgG concentrations 24 hr after tracer injection.

FIGS. 7A and 7B are graphs of data that show that administration of DVN24 but not ADM32 mAbs reduces the serum concentration of hIgG but not HSA.

FIG. 7A: Clearance of hIgG.

FIG. 7B: Clearance of HSA. 1000 μg of tracer hIgG and HSA was injected intraperitoneally into groups of 3 mouse FcRn−/− hFcRn (line 276 transgenic) mice on day 0. 1000 μg of DVN24, ADM31, or negative control mAb was injected intraperitoneally on days 2, 3 and 4. Sera from serial eye bleeds were then analyzed by ELISA for the concentration of hIgG tracer. The mean±standard error values are based on the percent of serum tracer hIgG concentrations remaining relative to concentrations 24 hr after tracer injection. Comparisons of p<0.05 are indicated (*).

FIGS. 8A and 8B are graphs of data that show that DVN24 reduces arthritic lesions caused by human rheumatoid arthritis plasma. Groups of 3 mFcRn−/− Fcgr2b−/− hFcRn transgenic (line 32) mice were injected intraperitoneally with 0.5, 1, and 1 ml of human RA plasma on days 0, 2 and 7, respectively, and also injected intraperitoneally with 1 mg of purified DVN24 or isotype control IgGa mAbs on days 1, 3 and 8. Ankle width and overall arthritis scores were measured in a blinded manner by two independent observers, as described (Akilesh et al., 2004, J Clin Invest 113: 1328-33). Data are the mean±standard error. Comparisons of p<0.05 (*) and (p<0.005) (**) of the ankle widths are indicated.

### DETAILED DESCRIPTION OF THE INVENTION

Certain aspects of the present invention are based, at least in part, on the finding that the receptor FcRn (FcRp/Fcgrt1) selectively protects antibodies of the IgG isotype from normal protein catabolism in a Fc-dependent manner. FcRn is a novel member of a family of proteins that perform varied immunological functions. The FcRn molecule is expressed in the vascular endothelium along with other tissues of adult animals, including mice and humans. FcRn binds to antibody molecules, but only those from the IgG class. The crystal structures of the FcRn/IgG complex have been solved (Bjorkman and Simister, 1992, PNAS 89:638-42; West and Bjorkman, 2000, Biochemistry 39:9698-9708), proving that a receptor/ligand relationship exists between the two molecules. Further, FcRn heterodimerizes with (β2-microglobulin, and the (β2-microglobulin complex is critical for FcRn to bind to IgG in a pH-dependant manner.

Most serum proteins have a short serum half-life (about 1-2 days). However, two types of serum proteins, albumin and antibodies of the IgG class, have greatly extended serum half-lives. Their half-lives are extended because they are naturally rescued from normal catabolic degradation by the major histocompatibility complex family protein, FcRn. Several investigators have indirectly demonstrated a protective effect by coupling the Fc region of IgG to different polypeptides to improve stability of the polypeptide (e.g., U.S. Pat. Nos. 6,096,871 and 6,121,022). PCT Application WO 97/34631 also describes the use of immunoglobulin-like domains in increasing the stability and longevity of pharmaceutical compositions for therapeutic and diagnostic purposes. In addition, Applicants have shown that the genetic elimination of FcRn by gene targeting protects K/BxN mice from developing autoimmune arthritis (Akilesh et al., 2004, J Clin Invest 113: 1328-33. Applicants have also shown that genetic elimination of FcRn by gene targeting reduces the severity of systemic lupus erythematosus (SLE) in mice genetically predisposed to develop SLE-like disease. Applicant and others have suggested that the functional saturation of the FcRn protection pathway results in an amelioration of arthritis and in immune thrombocytopenic purpura mouse models (Akilesh et al., 2004, J Clin Invest 113: 1328-33; Hanson and Balthasar, 2002, Thromb Haemo 88: 898-899) in pathogenic serum transfer models. Thus, these experiments suggest that FcRn is a promising therapeutic target to treat autoimmune diseases such as those caused by autoantibodies. Recent studies by Applicants and their collaborators (e.g., Chaudhury et al., 2003, J Exp Med 197: 315-322) have shown that FcRn also protects albumin from normal catabolic elimination. This occurs because FcRn binds albumin and protects it from normal catabolic elimination in a similar manner as found for IgG. A major complication to the strategy of therapeutic blockade of FcRn protection of IgG is that such therapeutics could also reduce the serum half-life of albumin. This may result in deleterious side effects since maintenance of a normal serum concentration of albumin is critical for the maintenance of normal osmolarity and other biological functions for which albumin plays an essential role. To avoid this potentially serious side effect of anti-FcRn therapeutics, certain embodiments of the invention provide anti-FcRn therapeutics that are designed to selectively decrease the serum half-life of IgG but not the serum half-life of human albumin.

### I. FcRn Antibodies and Other FcRn Binding Agents

This invention provides, in part, FcRn binding agents that selectively target portions of the FcRn molecule, such as, for example, FcRn antibodies, antigen binding portions of FcRn antibodies, and non-immunoglobulin binding agents of FcRn. The FcRn binding agents described herein may be used to treat a variety of disorders, particularly FcRn-related autoimmune diseases. The invention provides antibodies and antigen binding portions thereof that modulate (inhibit or enhance) FcRn mediated functions, such as Fc binding or IgG protection activities. Such binding agents may be used to modulate FcRn functions in vitro and in vivo, and, in particular, for treating FcRn-related autoimmune diseases. In particular embodiments, the present invention relates to monoclonal antibodies against FcRn.

In one embodiment, FcRn antibodies (immunoglobulins) are raised against an isolated and/or recombinant human FcRn or portion thereof (e.g., peptide) or against a host cell which expresses recombinant human FcRn. As used herein, the term “FcRn,” also referred to in the literature as FcRn alpha chain, refers to an FcRn polypeptide from a mammal including, for example, a human. In certain aspects, antibodies of the invention specifically bind to a region of an FcRn protein (e.g., the alpha 2 domain helix), which constitutes an Fc binding site (see, e.g., West and Bjorkman, 2000, Biochemistry 39:9698-9708). In other cases, antibodies of the invention specifically bind to a region of an FcRn protein that constitutes a β2-microglobulin binding site. Antibodies of the invention inhibit binding of FcRn to IgG but do not inhibit binding of FcRn to human albumin.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure: they include relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol., 1997, 196:901-917; Chothia et al. Nature, 1989, 342:878-883 (1989).

As used herein, the term “antibody” refers to an intact immunoglobulin or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms “anti-FcRn antibody” and “FcRn antibody” are used interchangeably herein.

An Fab fragment is a monovalent fragment consisting of the VL, VH, CL and CH I domains; a F(ab′)₂ fragment is a bivalent fragment comprising two Fab fragments linked by a disulfide bridge at the hinge region; an Fd fragment consists of the VH and CH1 domains; an Fv fragment consists of the VL and VH domains of a single arm of an antibody; and a dAb fragment (Ward et al., Nature 341:544-546, 1989) consists of a VH domain.

A single-chain antibody (scFv) is an antibody in which a VL and VH regions are paired to form a monovalent molecules via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444-6448, 1993, and Poljak, R. J., et al., Structure 2:1121-1123, 1994). One or more CDRs may be incorporated into a molecule either covalently or noncovalently.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, as described below.

The term “chimeric antibody” refers to an antibody that contains one or more regions from one antibody and one or more regions from one or more other different antibodies. In one embodiment, one or more of the CDRs are derived from a human anti-FcRn antibody. In a more preferred embodiment, all of the CDRs are derived from a human anti-FcRn antibody. In another preferred embodiment, the CDRs from more than one human anti-FcRn antibodies are mixed and matched in a chimeric antibody. For instance, a chimeric antibody may comprise a CDR1 from the light chain of a first human anti-FcRn antibody combined with CDR2 and CDR3 from the light chain of a second human anti-FcRn antibody, and the CDRs from the heavy chain may be derived from a third anti-FcRn antibody. Further, the framework regions may be derived from one of the same anti-FcRn antibodies, from one or more different antibodies, such as a human antibody, or from a humanized antibody.

In certain embodiments, the FcRn antibody or antigen binding portion thereof is linked to an additional functional moiety. Such linkage may be covalent or non-covalent. In one embodiment, the functional moiety may be therapeutic, e.g., a drug conjugate or toxin.

In certain further embodiments, the FcRn antibody or antigen binding portion thereof is labeled to facilitate detection. As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, beta-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

As shown in the Examples below, Applicants have generated monoclonal antibodies against human FcRn, as well as hybridoma cell lines producing FcRn monoclonal antibodies. These antibodies were further characterized in many ways, for example, their ability to inhibit interaction between human FcRn and its ligands (e.g., human IgG or human serum albumin), their ability to decrease the serum half-life of IgG in vivo, their ability to promote clearance of IgG in vivo, and their ability to ameliorate the inflammatory lesions induced by pathogenic human antibodies. The FcRn antibodies that specifically bind to human IgG, but do not bind to human serum albumin (HSA) are particularly useful for therapeutic purposes.

In certain embodiments, antibodies of the invention specifically-bind to an extracellular domain (ECD) of an FcRn protein (also referred to herein as a soluble FcRn polypeptide). A representative soluble FcRn polypeptide may comprise amino acids residues 24-297 of SEQ ID NO: 1 below. As used herein, the FcRn soluble polypeptides include fragments, functional variants, and modified forms of FcRn soluble polypeptide.

(SEQ ID NO: 1)
mgvprpqpwa lglllfllpg slgaeshlsl lyhltavssp
apgtpafwvs gwlgpqqyls
ynslrgeaep cgawvwenqv swywekettd lrikeklfle
afkalggkgp ytlqgllgce
lgpdntsvpt akfalngeef mnfdlkqgtw ggdwpealai
sqrwqqqdka ankeltfllf
scphrlrehl ergrgnlewk eppsmrlkar psspgfsvlt
csafsfyppe lqlrflrngl
aagtgqgdfg pnsdgsfhas ssltvksgde hhyccivqha
glaqplrvel espakssvlv
vgivigvlll taaavggall wrrmrsglpa pwislrgddt
gvllptpgea qdadlkdvnv
ipata

In certain embodiments, the present invention provides monoclonal FcRn antibodies that specifically bind an FcRn or a portion of FcRn. Examples of the monoclonal FcRn antibodies include, but are not limited to, DVN21 and DVN24 as described below in the working examples. In certain embodiments, the immunoglobulins bind to FcRn with an affinity of at least about 1×10⁻⁶, 1×10⁻⁷, 1×10⁻⁸, 1×10⁻⁹ M or less.

In certain aspects of the invention, anti-FcRn antibodies of the invention demonstrate both molecule and species selectivity. For example, antibodies disclosed herein are preferably specific for FcRn, with minimal binding to other FcRn ligand molecules, such as, for example, HSA. In one embodiment, the anti-FcRn antibody binds to human, cynomologous or rhesus FcRn. In one embodiment, the anti-FcRn antibody does not bind to mouse, rat, guinea pig, dog, goat or rabbit FcRn. Alternatively, the antibody binds to more than one different FcRn molecules from different species, such as human and mouse. Following the teachings of the specification, one may determine the molecule and species selectivity for the anti-FcRn antibody using methods well known in the art, for example, immunofluorescence microscopy, Western blot, FACS, ELISA or RIA. In one embodiment, the anti-FcRn antibody has a tendency to bind to FcRn that is at least 50 times greater than its tendency to bind to other FcRn ligand molecules, and preferably 100 or 200 times greater.

In certain embodiments, antibodies of the present invention bind to one or more specific domains of FcRn. For example, a subject antibody binds to a region in the Fe-binding site of the FcRn heavy chain.

The anti-FcRn antibody may be an IgG, an IgM, an IgE, an IgA or an IgD molecule. In a preferred embodiment, the antibody is an IgG and is an IgG1, IgG2, IgG3 or IgG4 subtype. In an specific embodiment, the anti-FcRn antibody is subclass IgG2. The class and subclass of FcRn antibodies may be determined by any method known in the art. In general, the class and subclass of an antibody may be determined using antibodies that are specific for a particular class and subclass of antibody. Such antibodies are available commercially. The class and subclass can be determined by ELISA, Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In certain embodiments, single chain antibodies, and chimeric, humanized or primatized (CDR-grafted) antibodies, as well as chimeric or CDR-grafted single chain antibodies, comprising portions derived from different species, are also encompassed by the present invention as antigen binding portions of an FcRn antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023; Boss et al., U.S. Pat. No. 4,816,397; Boss et al., European Patent No. 0,120,694; Neuberger, M. S. et al., WO 86/01533; Neuberger, M. S. et al., European Patent No. 0,194,276 B1; Winter, U.S. Pat. No. 5,225,539; and Winter, European Patent No. 0,239,400 B1. See also, Newman, R. et al., BioTechnology, 10: 1455-1460 (1992), regarding primatized antibody. See, e.g., Ladner et al., U.S. Pat. No. 4,946,778; and Bird, R. E. et al., Science, 242: 423-426 (1988)), regarding single chain antibodies.

In addition, functional fragments of antibodies, including fragments of chimeric, humanized, primatized or single chain antibodies, can be produced. Functional fragments of the subject antibodies retain at least one binding function and/or modulation function of the full-length antibody from which they are derived. Preferred functional fragments retain an antigen binding function of a corresponding full-length antibody (e.g., specificity for an FcRn). Certain preferred functional fragments retain the ability to inhibit one or more functions characteristic of an FcRn, such as a binding activity or a transport activity. For example, in one embodiment, a functional fragment of an FcRn antibody can specifically inhibit the interaction of FcRn with one of its ligands (e.g., IgG) and/or can inhibit one or more FcRn-mediated functions in vivo, such as IgG transport and autoimmune responses.

In certain embodiments, antibody fragments that bind to an FcRn receptor or portion thereof, including, but not limited to, Fv, Fab, Fab′ and F(ab′)₂ fragments are encompassed by the invention. Such fragments can be produced by enzymatic cleavage or by recombinant techniques. For instance, papain or pepsin cleavage can generate Fab or F(ab′)₂ fragments, respectively. Antibodies can also be produced in a variety of truncated forms using antibody-encoding genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

A humanized antibody can be, for example, an antibody that is derived from a non-human species, in which certain amino acids in the framework and constant domains of the heavy and light chains have been mutated so as to reduce of abolish an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293. A humanized antibody may comprise portions of immunoglobulins of different origin. For example, at least one portion can be of human origin. Accordingly, the present invention relates to a humanized immunoglobulin having binding specificity for an FcRn (e.g., human FcRn), said immunoglobulin comprising an antigen binding region of nonhuman origin (e.g., rodent) and at least a portion of an immunoglobulin of human origin (e.g., a human framework region, a human constant region or portion thereof). For example, the humanized antibody can comprise portions derived from an immunoglobulin of nonhuman origin with the requisite specificity, such as a mouse, and from immunoglobulin sequences of human origin (e.g., a chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain).

Another example of a humanized immunoglobulin of the present invention is an immunoglobulin containing one or more immunoglobulin chains comprising a CDR of nonhuman origin (e.g., one or more CDRs derived from an antibody of nonhuman origin) and a framework region derived from a light and/or heavy chain of human origin (e.g., CDR-grafted antibodies with or without framework changes). In one embodiment, the humanized immunoglobulin can compete with murine monoclonal antibody for binding to an FcRn polypeptide. Chimeric or CDR-grafted single chain antibodies are also encompassed by the term humanized immunoglobulin.

In certain embodiments, the present invention provides FcRn antagonist antibodies. As described herein, the term “antagonist antibody” refers to an antibody that can inhibit one or more functions of an FcRn, such as a binding activity (e.g., ligand binding and β2-microglobin binding) or a transport activity (e.g., transporting IgG and protecting IgG from lysosomal catabolism).

In certain embodiments, anti-idiotypic antibodies are also provided. Anti-idiotypic antibodies recognize antigenic determinants associated with the antigen-binding site of another antibody. Anti-idiotypic antibodies can be prepared against a second antibody by immunizing an animal of the same species, and preferably of the same strain, as the animal used to produce the second antibody. See e.g., U.S. Pat. No. 4,699,880. In one embodiment, antibodies are raised against FcRn or a portion thereof, and these antibodies are used in turn to produce an anti-idiotypic antibody. The anti-idiotypic antibodies produced thereby can bind compounds which bind receptor, such as ligands of receptor function, and can be used in an immunoassay to detect or identify or quantitate such compounds. Such an anti-idiotypic antibody can also be an inhibitor of an FcRn receptor function, although it does not bind receptor itself. Such an anti-idiotypic antibody can also be called an antagonist antibody.

In certain aspects, the present invention relates to hybridoma cell lines, as well as to monoclonal antibodies produced by these hybridoma cell lines. The cell lines of the present invention have uses other than for the production of the monoclonal antibodies. For example, the cell lines of the present invention can be fused with other cells (such as suitably drug-marked human myeloma, mouse myeloma, human-mouse heteromyeloma or human lymphoblastoid cells) to produce additional hybridomas, and thus provide for the transfer of the genes encoding the monoclonal antibodies. In addition, the cell lines can be used as a source of nucleic acids encoding the anti-FcRn immunoglobulin chains, which can be isolated and expressed (e.g., upon transfer to other cells using any suitable technique (see e.g., Cabilly et al., U.S. Pat. No. 4,816,567; Winter, U.S. Pat. No. 5,225,539)). For instance, clones comprising a rearranged anti-FcRn light or heavy chain can be isolated (e.g., by PCR) or cDNA libraries can be prepared from mRNA isolated from the cell lines, and cDNA clones encoding an anti-FcRn immunoglobulin chain can be isolated. Thus, nucleic acids encoding the heavy and/or light chains of the antibodies or portions thereof can be obtained and used in accordance with recombinant DNA techniques for the production of the specific immunoglobulin, immunoglobulin chain, or variants thereof (e.g., humanized immunoglobulins) in a variety of host cells or in an in vitro translation system. For example, the nucleic acids, including cDNAs, or derivatives thereof encoding variants such as a humanized immunoglobulin or immunoglobulin chain, can be placed into suitable prokaryotic or eukaryotic vectors (e.g., expression vectors) and introduced into a suitable host cell by an appropriate method (e.g., transformation, transfection, electroporation, infection), such that the nucleic acid is operably linked to one or more expression control elements (e.g., in the vector or integrated into the host cell genome). For production, host cells can be maintained under conditions suitable for expression (e.g., in the presence of inducer, suitable media supplemented with appropriate salts, growth factors, antibiotic, nutritional supplements, etc.), whereby the encoded polypeptide is produced. If desired, the encoded protein can be recovered and/or isolated (e.g., from the host cells or medium). It will be appreciated that the method of production encompasses expression in a host cell of a transgenic animal (see e.g., WO 92/03918, GenPharm International, published Mar. 19, 1992).

### II. Methods of Antibody Production

Preparation of immunizing antigen, and polyclonal and monoclonal antibody production can be performed as described herein, or using other suitable techniques. A variety of methods have been described. See e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991). Generally, a hybridoma can be produced by fusing a suitable immortal cell line (e.g., a myeloma cell line such as SP2/0) with antibody producing cells. The antibody producing cell, preferably those of the spleen or lymph nodes, are obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity can used, including, for example, methods which select recombinant antibodies from a library, or which rely upon immunization of transgenic animals (e.g., mice) capable of producing a full repertoire of human antibodies. See e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807. For example, FcRn antibodies may be isolated from a synthetic human combinatorial antibody library (HuCAL). See, e.g., Knappik et al., 2000, J Mol boil 296:57-86.

To illustrate, immunogens derived from an FcRn polypeptide (e.g., an FcRn polypeptide or an antigenic fragment thereof which is capable of eliciting an antibody response, or an FcRn fusion protein) can be used to immunize a mammal, such as a mouse, a hamster or rabbit. See, for example, Antibodies: A Laboratory Manual ed. by Harlow and Lane (Cold Spring Harbor Press: 1988). Techniques for conferring immunogenicity on a protein or peptide include conjugation to carriers or other techniques well known in the art. An immunogenic portion of an FcRn polypeptide can be administered in the presence of adjuvant. The progress of immunization can be monitored by detection of antibody titers in plasma or serum. Standard ELISA or other immunoassays can be used with the immunogen as antigen to assess the levels of antibodies. In one embodiment, antibodies of the invention are specific for the extracellular portion of an FcRn protein or fragments thereof. In another embodiment, antibodies of the invention are specific for the intracellular portion or the transmembrane portion of the FcRn protein.

Following immunization of an animal with an antigenic preparation of an FcRn polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an FcRn polypeptide and monoclonal antibodies isolated from a culture comprising such hybridoma cells.

In certain embodiments, antibodies of the present invention can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments.

In certain embodiments, antibodies of the present invention are further intended to include bispecific, single-chain, and chimeric and humanized molecules having affinity for an FcRn polypeptide conferred by at least one CDR region of the antibody. Techniques for the production of single chain antibodies (U.S. Pat. No. 4,946,778) can also be adapted to produce single chain antibodies. Also, transgenic mice or other organisms including other mammals, may be used to express humanized antibodies. Methods of generating these antibodies are known in the art. See, e.g., Cabilly et al., U.S. Pat. No. 4,816,567; Cabilly et al., European Patent No. 0,125,023; Queen et al., European Patent No. 0,451,216; Boss et al., U.S. Pat. No. 4,816,397; Boss et al., European Patent No. 0,120,694; Neuberger, M. S. et al., WO 86/01533; Neuberger, M. S. et al., European Patent No. 0,194,276; Winter, U.S. Pat. No. 5,225,539; winter, European Patent No. 0,239,400; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)).

Such humanized immunoglobulins can be produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993)).

In certain embodiments, the antibodies are further attached to a label that is able to be detected (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. A binding agent affixed to such a moiety may be used as an imaging agent and is administered in an amount effective for diagnostic use in a mammal such as a human and the localization and accumulation of the imaging agent is then detected. The localization and accumulation of the imaging agent may be detected by radioscintigraphy, nuclear magnetic resonance imaging, computed tomography or positron emission tomography. Immunoscintigraphy using antibodies or other binding polypeptides directed at FcRn may be used to detect and/or diagnose cancers and vasculature. For example, monoclonal antibodies against the FcRn marker labeled with ⁹⁹Technetium, ¹¹¹Indium, ¹²⁵Iodine—may be effectively used for such imaging. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, preferably 1-10 millicuries, most often 2-5 millicuries are administered. Thus, compositions according to the present invention useful as imaging agents comprising a targeting moiety conjugated to a radioactive moiety comprise 0.1-100 millicuries, in some embodiments preferably 1-10 millicuries, in some embodiments preferably 2-5 millicuries, in some embodiments more preferably 1-5 millicuries.

In certain preferred embodiments, an antibody of the invention is a monoclonal antibody, and in certain embodiments the invention makes available methods for generating novel antibodies. For example, a method for generating a monoclonal antibody that binds specifically to an FcRn polypeptide may comprise administering to a mouse an amount of an immunogenic composition comprising the FcRn polypeptide effective to stimulate a detectable immune response, obtaining antibody-producing cells (e.g., cells from the spleen) from the moose and fusing the antibody-producing cells with myeloma cells to obtain antibody-producing hybridomas, and testing the antibody-producing hybridomas to identify a hybridoma that produces a monoclonal antibody that binds specifically to the FcRn polypeptide. Once obtained, a hybridoma can be propagated in a cell culture, optionally in culture conditions where the hybridoma-derived cells produce the monoclonal antibody that binds specifically to FcRn polypeptide. The monoclonal antibody may be purified from the cell culture.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g., by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g., the Biacore binding assay, Bia-core AB, Uppsala, Sweden), sandwich assays (e.g., the paramagnetic bead system of IGEN International, Inc., Gaithersburg, Md.), western blots, immunoprecipitation assays and immunohistochemistry.

The antibodies of the present invention are useful in a variety of applications, including research, diagnostic and therapeutic applications. For instance, they can be used to isolate and/or purify receptor or portions thereof, and to study receptor structure (e.g., conformation) and function.

### III. Diagnostic Applications

In certain aspects, the FcRn antibodies of the present invention can be used to detect or measure the expression of FcRn receptor, for example, in endogenous cells which express FcRn (e.g., intestinal epithelial cells and vascular endothelial cells), or in cells transfected with an FcRn receptor gene. Detection of FcRn in a patient or individual may be important in determining the extent of participation of FcRn plays in any autoimmune disease process. In certain embodiments, FcRn antibodies may have utility in cell imaging (e.g., flow cytometry, and fluorescence activated cell sorting) for diagnostic or research purposes.

In certain embodiments, the antibodies or antigen binding fragments of the antibodies can be labeled or unlabeled for diagnostic purposes. Typically, diagnostic assays entail detecting the formation of a complex resulting from the binding of an antibody to FcRn. The antibodies can be directly labeled. A variety of labels can be employed, including, but not limited to, radionuclides, fluorescers, enzymes, enzyme substrates, enzyme cofactors, enzyme inhibitors and ligands (e.g., biotin, haptens). Numerous appropriate immunoassays are known to the skilled artisan (see, for example, U.S. Pat. Nos. 3,817,827; 3,850,752; 3,901,654; and 4,098,876). When unlabeled, the antibodies can be used in assays, such as agglutination assays. Unlabeled antibodies can also be used in combination with another (one or more) suitable reagent which can be used to detect antibody, such as a labeled antibody (e.g., a second antibody) reactive with the first antibody (e.g., anti-idiotype antibodies or other antibodies that are specific for the unlabeled immunoglobulin) or other suitable reagent (e.g., labeled protein A). An FcRn antibody may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding.

In one embodiment, the antibodies of the present invention can be utilized in enzyme immunoassays, wherein the subject antibodies, or second antibodies, are conjugated to an enzyme. When a biological sample comprising an FcRn protein is combined with the subject antibodies, binding occurs between the antibodies and FcRn protein. In one embodiment, a sample containing cells expressing an FcRn protein (e.g., endothelial cells) is combined with the subject antibodies, and binding occurs between the antibodies and cells hearing an FcRn protein comprising an epitope recognized by the antibody. These bound cells can be separated from unbound reagents and the presence of the antibody-enzyme conjugate specifically bound to the cells can be determined, for example, by contacting the sample with a substrate of the enzyme which produces a color or other detectable change when acted on by the enzyme. In another embodiment, the subject antibodies can be unlabeled, and a second, labeled antibody can be added which recognizes the subject antibody.

In certain aspects, kits for use in detecting the presence of an FcRn protein in a biological sample can also be prepared. Such kits will include an antibody which binds to an FcRn protein or portion of said receptor, as well as one or more ancillary reagents suitable for detecting the presence of a complex between the antibody and FcRn or portion thereof. The antibody compositions of the present invention can be provided in lyophilized form, either alone or in combination with additional antibodies specific for other epitopes. The antibodies, which can be labeled or unlabeled, can be included in the kits with adjunct ingredients (e.g., buffers, such as Tris, phosphate and carbonate, stabilizers, excipients, biocides and/or inert proteins, e.g., bovine serum albumin). For example, the antibodies can be provided as a lyophilized mixture with the adjunct ingredients, or the adjunct ingredients can be separately provided for combination by the user. Generally these adjunct materials will be present in less than about 5% weight based on the amount of active antibody, and usually will be present in a total amount of at least about 0.001% weight based on antibody concentration. Where a second antibody capable of binding to the monoclonal antibody is employed, such antibody can be provided, in the kit, for instance in a separate vial or container. The second antibody, if present, is typically labeled, and can be formulated in an analogous manner with the antibody formulations described above.

Similarly, the present invention also relates to a method of detecting and/or quantitating expression of an FcRn or portion of the receptor by a cell, wherein a composition comprising a cell or fraction thereof (e.g., membrane fraction) is contacted with an antibody which binds to an FcRn or portion of the receptor under conditions appropriate for binding of the antibody thereto, and antibody binding is monitored. Detection of the antibody, indicative of the formation of 3 complex between antibody and FcRn or a portion thereof, indicates the presence of the receptor. Binding of antibody to the cell can be determined by standard methods, such as those described in the working examples. The method can be used to detect expression of FcRn in cells from an individual. In certain embodiments, a quantitative expression of FcRn in endothelial cells can be evaluated, for instance, by immunofluorescence microscopy. In alternative embodiments, a quantitative assessment can be evaluated by flow cytometry of blood leukocytes and flow cytometry, and the staining intensity can be correlated with disease susceptibility, progression or risk.

The present invention also relates to a method of detecting the susceptibility of a mammal to certain diseases. To illustrate, the method can be used to detect the susceptibility of a mammal to diseases which progress based on the amount of FcRn present on cells and/or the number of FcRn-positive cells in a mammal. In one embodiment, the invention relates to a method of detecting susceptibility of a mammal to an autoimmune disease. In this embodiment, a sample to be tested is contacted with an antibody which binds to an FcRn or portion thereof under conditions appropriate for binding of said antibody thereto, wherein the sample comprises cells which express FcRn in normal individuals. The binding of antibody and/or amount of binding is detected, which indicates the susceptibility of the individual to an autoimmune disease, wherein higher levels of receptor correlate with increased susceptibility of the individual to an autoimmune disease.

### IV. Therapeutic Applications

In certain embodiments, the present invention provides compositions and methods for treating autoimmune diseases (or disorders). In other embodiments, the present invention provides methods for inhibiting FcRn-mediated IgG protection in an individual. These methods involve administering to the individual a therapeutically effective amount of one or more FcRn antibodies as described above. These methods are particularly aimed at therapeutic and prophylactic treatments of animals, and more particularly, humans.

As described herein, autoimmune diseases suitable for treatment by the FcRn antibodies include, but are not limited to, systemic lupus erythematosus, insulin resistant diabetes, myasthenia gravis, polyarteritis, autoimmune thrombocytopenic purpura, cutaneous vasculitis, bullous pemphigoid, pemphigus vulgaris, pemphigus foliaceus, Goodpasture's syndrome, rheumatoid arthritis, Kawasaki's disease, and Sjogren's syndrome.

In certain embodiments of such methods, one or more FcRn antibodies can be administered, together (simultaneously) or at different times (sequentially). In a specific embodiment, the subject antibodies of the present invention can also be used with other antibody therapeutics (monoclonal or polyclonal).

In certain embodiments, the FcRn antibodies of the invention can be used alone. Alternatively, the FcRn antibodies may be used in combination with an immunostimulatory agent, an immunomodulator, or a combination thereof. A wide array of conventional compounds have been shown to have immunomodulating activities, including but not limited to, alpha-interferon, gamma-interferon, tumor necrosis factor-alpha, or a combination thereof. The present invention recognizes that the effectiveness of conventional therapies for autoimmune diseases, which can be enhanced through the use of one or more FcRn antibodies of the invention.

in certain embodiments, the FcRn antibodies of the invention can be combined with a known therapy for autoimmune diseases. Examples of such known therapies for autoimmune diseases include, but are not limited to, therapies with nonsteroidal anti-inflammatory drugs (NSAID) or corticosteroids. Other examples of therapies for autoimmune diseases include periodic administration of patients with high doses of antibodies.

In certain particular embodiments, the FcRn antibodies of the invention can be employed to promote clearance of radioactive antibodies or antibody conjugated toxins used for imaging or treatment of cancer.

Depending on the nature of the combinatory therapy, administration of the antibodies of the invention may be continued while the other therapy is being administered and/or thereafter. Administration of the antibodies may be made in a single dose, or in multiple doses. In some instances, administration of the antibodies is commenced at least several days prior to the conventional therapy, while in other instances, administration is begun either immediately before or at the time of the administration of the conventional therapy.

### V. Pharmaceutical Compositions and Modes of Administration

In certain embodiments, the subject antibodies of the present invention are formulated with a pharmaceutically acceptable carrier. Such antibodies can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject antibodies include those suitable for oral, dietary, topical, parenteral (e.g., intravenous, intraarterial, intramuscular, subcutaneous injection), inhalation (e.g., intrabronchial, intranasal or oral inhalation, intranasal drops), rectal, and/or intravaginal administration. Other suitable methods of administration can also include rechargeable or biodegradable devices and slow release polymeric devices. The pharmaceutical compositions of this invention can also be administered as part of a combinatorial therapy with other agents (either in the same formulation or in a separate formulation).

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of immune-modulating agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of one or more subject antibodies as an active ingredient.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Methods of the invention can be administered topically, for example, to skin. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The subject antibodies may be mixed under sterile conditions with a pharmaceutically acceptable carrier, and with any preservatives, buffers, or propellants which may be required. The ointments, pastes, creams and gels may contain, in addition to an antibody, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Pharmaceutical compositions suitable for parenteral administration may comprise one or more antibodies in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more antibodies in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

### Exemplification

The invention now being generally described, it will be more readily understood by reference to the following examples, which are included merely for purposes of illustration of certain aspects and embodiments of the present invention, and are not intended to limit the invention.

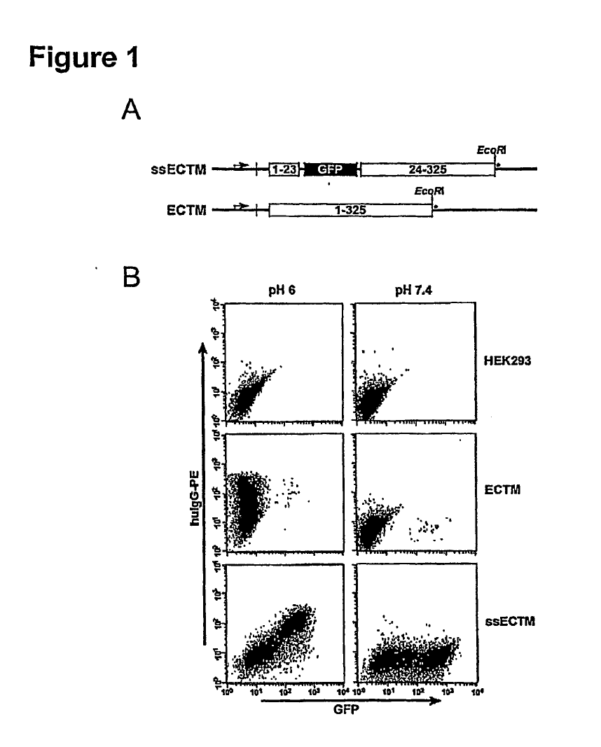

Applicants' first goal was to produce a cell line in which hIgG and albumin binding to hFcRn could be measured conveniently using cell surface monitoring methods, such as flow cytometry. The steady state localization of FcRn is normally endosomal (Claypool et al., 2002, J Biol Chem 277: 28038-50; Ober et al., 2004, J Immunol 172: 2021-9). To facilitate visualization of hFcRn, Applicants produced a construct with a green fluorescent protein (GFP)—encoding cDNA fragment cloned in-frame between the terminal signal sequence codon and the first codon of the mature hFcRn protein. To divert hFcRn from the endosomes to the plasma membrane, Applicants then engineered the construct so that the normal cytoplasmic endosomal targeting domain was deleted (FIG. 1A). When transfected into human HEK293 cells, the GFP-modified construct (ssECTM) and a similar construct lacking GFP (ECTM) diverted hFcRn from the normal endosomal pattern to the plasma membrane.

ssECTM and ECTM constructs stably transfected into HEK293 cells were then used for flow cytometric analysis to analyze their ability to bind hIgG in a pH-dependent manner (FIG. 1B). The ssECTM and ECTM transfectants demonstrated equivalent hIgG binding at pH 6, but not pH 7.4, indicating that the GFP tag did not influence pH-dependent binding of hIgG to hFcRn. These results validated the use of the ssECTM transfected HEK293 cell line to monitor for hIgG and HSA binding.

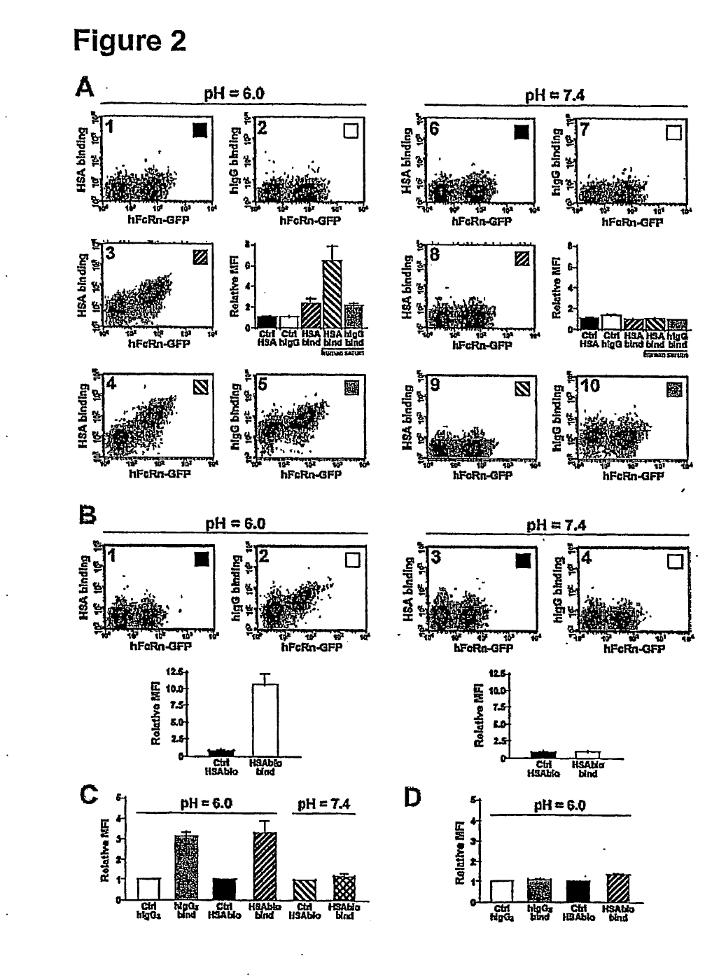

Flow cytometry was then used to assess the possible interaction of HSA, along with hIgG, with hFcRn (FIG. 2). Incubation of ssECTM cells at pH 6 with both purified HSA (FIG. 2A3; p≦0.002) and human serum (FIG. 2A4; p≦0.002) resulted in HSA binding detected by GAHbio in conjunction with SA-APC, while no binding was observed when ssECTM cells were incubated with GAHSA-biotin+SA-APC (FIG. 2A1) alone. At pH 6.0, it was similarly possible to detect hIgG binding (FIG. 2A5; p≦0.003) to hFcRn-GFP when ssECTM cells were pre-incubated with human serum and GAH IgG-PE, while no binding was detected when cells were incubated only with GAH IgG-PE (FIG. 2A2). At neutral pH (pH 7.4), no binding of IgG or HSA to ssECTM cells expressing hFcRn-GFP was observed (FIG. 2A6-10).

To corroborate the specificity of HSA binding, Applicants performed similar experiment by using biotinylated HSA (HSA-bio; FIG. 2B). At an acidic pH, binding of HSA-bio to ssECTM cells was observed when detected with SA-APC (FIG. 2B2; p≦0.001), as compared to SA-APC alone (FIG. 2B1). No binding was observed at neutral pH (FIGS. 2B3&4). Similar results were obtained by using ECTM cells (lacking the GFP tag), confirming that both HSA-bio (p≦0.006) and hIgG₃-AF₆₄₇ (p≦0.001) were able to bind specifically to hFcRn independent of the GFP tag (FIG. 2C). Thus, the acidic pH-dependent binding was not an artifact generated by the fusion of hFcRn and GFP. In addition, untransfected HEK 293 cells did not show appreciable HSAbio or hIgG₃-AF binding (FIG. 2D) at an acidic pH. These results validate the use of ssECTM-transfected cells for evaluating hIgG and HSA binding and suggested that hIgG and HSA hFcRn bind hFcRn at an acid (pH 6.0) but not neutral (pH 7.4) pH.

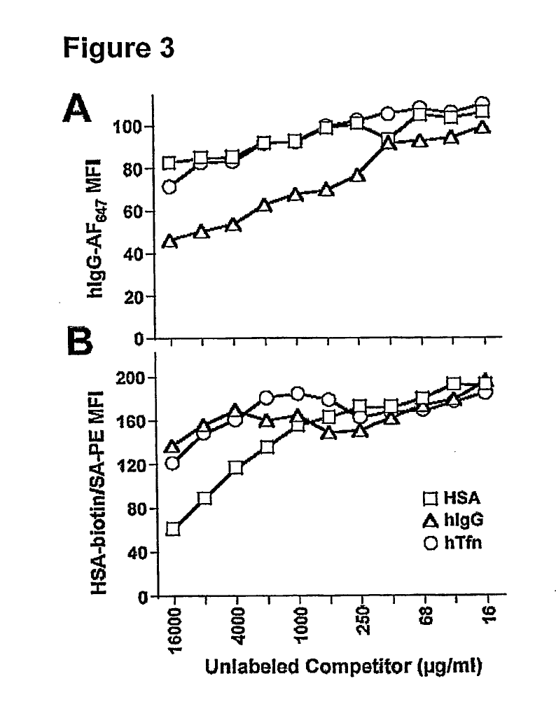

Having shown that both huIgG and HSA specifically bind to hFcRn in a pH dependent manner, Applicants then addressed whether there was overlap between the albumin and IgG binding sites of hFcRn. Applicants first evaluated the ability of HSA and hIgG, along with human transferrin (hTF), to inhibit binding of hIgG-AF and HSA-bio to ssECTM cells at pH 6. HSA and hTF failed to appreciably inhibit hIgG binding to hFcRn (FIG. 3A). Conversely, HSA inhibited hIgG-AF₆₄₇ binding minimally, only at high concentrations (>16 mg/ml), and no more than hTF (FIG. 3B). These results suggest that HSA and huIgG bind non-competing acid-pH-dependent sites on hFcRn.

Therapeutic blockade of FcRn is envisioned as a promising approach to treat autoimmune diseases caused by IgG autoantibodies (Christianson et al., 1996, J. Immunol. 176: 4933-39; Christianson et al., 1997, J Immunol 159: 4781-92; Liu et al., 1997, J Exp Med 186: 777-83; Akilesh et al., 2004, J Clin Invest 113: 1328-33). Indeed, mice deficient in FcRn are resistant to arthritis caused by pathogenic IgG antibodies (Akilesh et al., 2004, J Clin Invest 113: 1328-33). However, owing to the fact that hIgG and HSA bind hFcRn at an acid pH 6, a primary concern is that blockade of FcRn could result in the reduction of the T^{1/2} and the serum concentration of albumin. Since albumin is considered to be critical for the maintenance of normal colloid osmotic pressure, pH buffering and for transport of numerous molecules, including bile acids, fatty acids, vitamins and drugs (reviewed in Peters 1996, All About Albumin. New York, Academic Press), FcRn blockade could lead to serious side effects. To avoid such potentially serious side effects, anti-FcRn therapeutics would need to selectively inhibit IgG binding but not albumin.

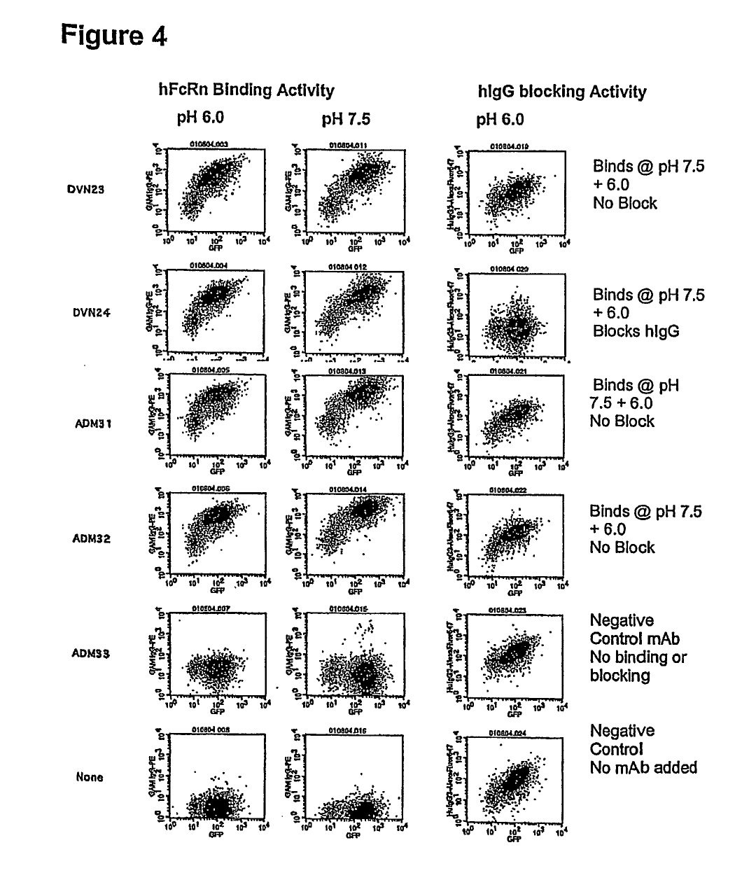

To determine whether it is possible to selectively block hIgG binding to hFcRn without impairing FcRn's binding and protection of albumin, Applicants generated a panel of monoclonal antibodies (mAbs) whose antigen combining site is specific for hFcRn. To do so, Applicants first immunized mice deficient in mouse FcRn with cells from mice expressing an hFcRn transgene As a primary goal was to identify mice producing antibodies capable of blocking the hIgG/hFcRn interaction, the sera were then screened using the ssECTM cell line in a flow cytometric assay to measure their ability to block hIgG from binding hFcRn at pH 6. Blocking activity was detected in sera of 14% of the immunized mice. Spleen cells of mice whose sera showed blocking activity were then immortalized using conventional hybridoma technology (Cooper and Paterson, 2004, Production of antibodies. Current Protocols in Immunology. New York, Wiley. 1: 2.4.1-2.5.14). Culture supernatants from growing hybridomas were then screened for pH 7.5 binding to hFcRn using a cellular ELISA described in the Materials and Methods. Supernatants from recloned hybridomas were similarly screened. As it was important for the invention that the mAbs secreted were able to bind hFcRn not only under neutral but also under acidic conditions, supernatants from stable hybridoma clones were then tested using ssECTM cells for their ability to bind hFcRn at pH 6. Purified mAbs ADM11, ADM12, DVN21, DVN23, DVN24, ADM 31 and ADM32 bound hFcRn in vitro at both pHs, while mAbs DVN1 and DVN22 bound hFcRn at pH 7.5 but not at pH 6.0. Example data for DVN 24, ADM31, ADM32 and a non-hFcRn specific control mAb ADM33 are shown in FIG. 4, left and center scattergrams.

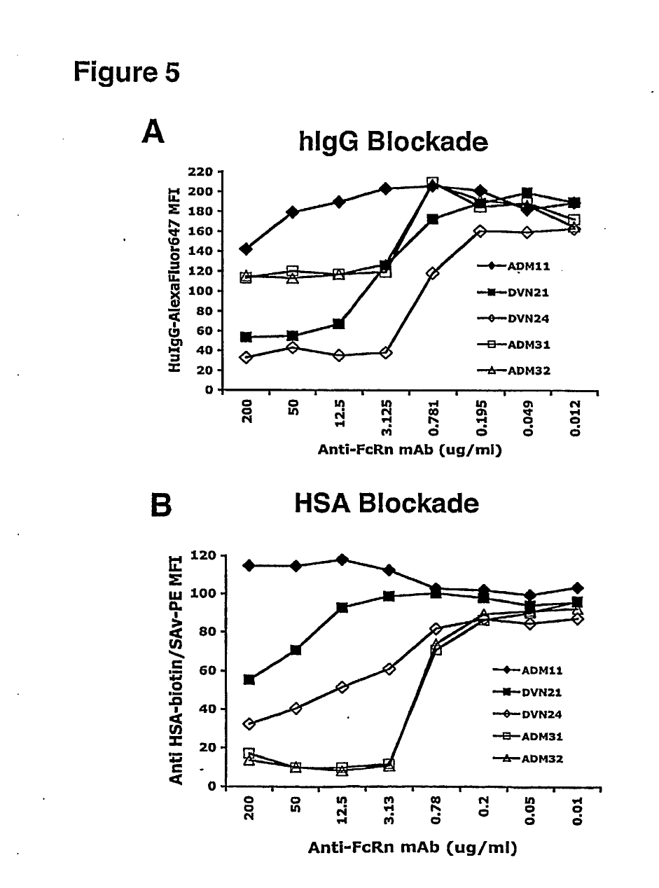

As a goal is the use of anti-FcRn mAbs for therapeutic blockade of hFcRn, the anti-hFcRn mAbs were then analyzed for their ability to block the binding of hFcRn at pH6 in vitro. A modification of the blocking assay used in FIG. 3A was used for this purpose. FIG. 4, far right scattergrams, shows data demonstrating that DVN24 effectively blocked the binding of hIgG3 to bFcRn, while none of the other anti-FcRn mAbs analyzed in this same experiment blocked binding of hIgG3 to hFcRn. FIG. 5A shows a compilation of flow cytometry data in which varying concentrations of several of the anti-hFcRn mAbs were used to determine their ability to block hIgG from binding hFcRn at pH 6. Only two mAbs, DVN21 and DVN24, showed effective blocking across a range of concentrations, and thus are candidates for the therapeutic blockade hFcRn, with DVN24 being most effective on a concentration basis.

However, for therapeutic application, it was critical that the mAbs capable of blocking the binding of hIgG did not also block albumin binding. Applicants therefore determined, using a modification of the blocking assay described in FIG. 3B, whether the panel of anti-hFcRn mAbs were capable of blocking the pH 6.0-dependent binding of HSA. As shown in FIG. 5B, increasing concentrations of DVN21 and DVN24 failed to appreciably inhibit the binding of albumin. In contrast, two anti-hFcRn mAbs, ADM31 and ADM32, effectively blocked the binding of HSA to hFcRn.

The fact that some anti-hFcRn mAbs effectively blocked the pH 6 dependent binding of hIgG, while others effectively blocked HSA binding strongly suggests that anti-hFcRn therapeutics can be developed which selectively target the IgG protection pathway while leaving the albumin protection pathway intact. It is thus envisioned that anti-hFcRn mAbs, exemplified by DVN21 and DVN24, would be excellent candidates for selective therapeutic blockade of hFcRn stabilizing the HSA in vivo.

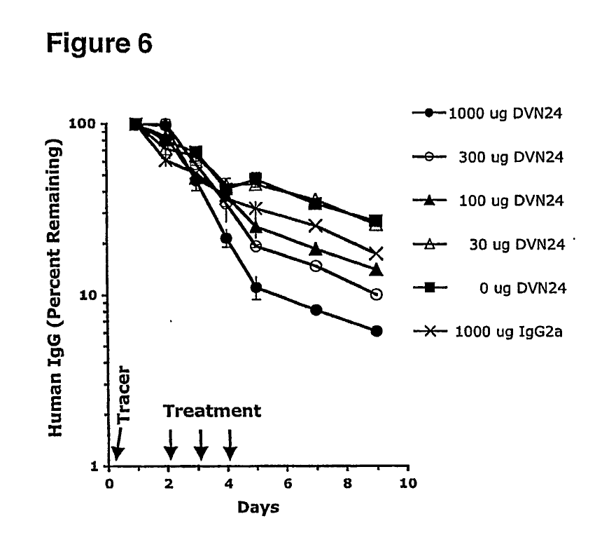

A primary goal of the invention is identify anti-FcRn mAbs that decrease the serum T^{1/2} of hIgG in vivo. To do so, Applicants produced mice lacking mouse FcRn but transgenic for hFcRn (Chaudhury et al., 2003, J Exp Med 197: 315-22; Roopenian et al., 2003, J Immunol 170: 3528-33). The extended T^{1/2} of hIgG compared with mice lacking mouse FcRn but not carrying the hFcRn transgene is a direct consequence of the bFcRn transgene (Roopenian et al., 2003, J Immunol 170: 3528-33). Applicants then tested whether the infusion of DVN24 was capable of therapeutically blocking hFcRn from stabilizing hIgG, resulting in a shortening of the serum T^{1/2} of previously administered hIgG tracer antibodies. FIG. 6 shows that increasing concentrations of infused DVN24 did indeed promote the clearance and thus decrease the T^{1/2} of the hIgG tracer in a dose dependent manner. The concentration of hIgG was reduced over 3-fold by day 9 compared with similar dose of a negative control isotype matched mAb.

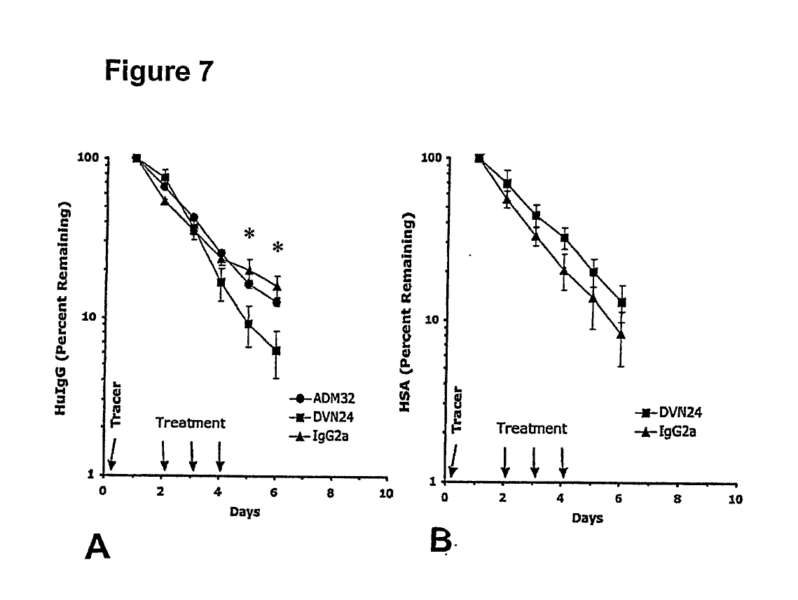

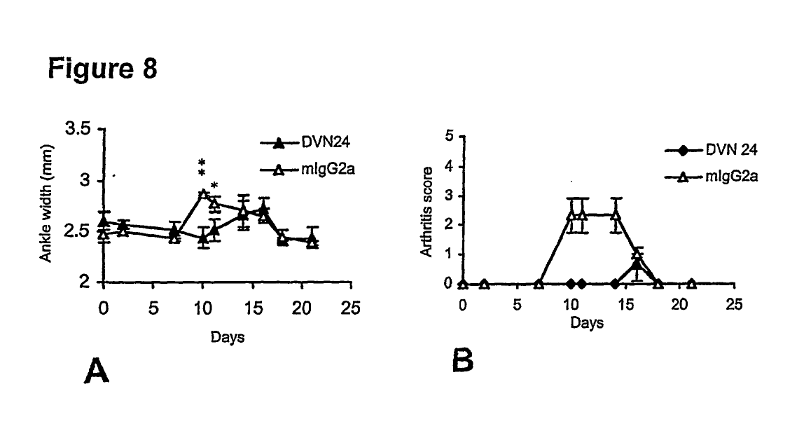

Applicants then compared the ability of DVN24 and ADM32 to promote the clearance of hIgG. DVN24 again promoted an approximately 3-fold reduction in the serum concentration of hIgG tracer at d6 (FIG. 7A). However, infusion with ADM32 failed significantly the influence the serum concentration of hIgG tracer beyond that accomplished by the negative control mAbs (FIG. 7A). Moreover DVN24 failed to significantly affect the concentration of HSA tracer (FIG. 8B). Since the in vitro blocking results (FIG. 6) indicated that ADM31 blocks HSA binding but not hIgG binding, and since DVN24 blocks hIgG but not HSA binding, these results indicate that it is possible to produce mAb blocking agents (exemplified by DVN24), which selectively increase the in vivo clearance of hIgG while not promoting the clearance of HSA. Such a block agent would thus be considered to be a prime candidate to deplete pathogenic autoantibodies without affecting serum albumin concentrations.

A key consideration toward the exploitation of anti-hFcRn therapeutics would be whether such therapeutics protect patients from autoimmune lesions. Indeed, Applicants have shown previously that a deficiency in mouse FcRn protects mice from developing arthritic joint lesions normally caused by the transfer of arthritogenic mouse IgG (Akilesh et al. 2004, J Clin Invest 113: 1328-33). However, it remained to be determined whether anti-hFcRn mAb therapeutics could be used to block human pathogenic autoantibodies. Applicants therefore developed a model in which IgG from patients with rheumatoid arthritis causes joint inflammation when transferred into mice genetically hypersensitized to develop humoral autoimmune disease because they are deficient in the inhibitory Fc receptor, Fcgr2b (Bolland and Ravetch, 2000, Immunity 13: 277-85; Akilesh et al. 2004, J Clin Invest 113: 1328-33). Applicants have found that sera or plasma from patients diagnosed with rheumatoid arthritis but not serum or plasma from undiseased controls causes transient ankle swelling and inflammation when transferred into Fcgr2b mice. The inflammatory activity was in the IgG fraction indicating that it was caused by IgG antibodies. To study whether the blockade of hFcRn by DVN24 could lead to amelioration of the joint lesions, Applicants produced mFcRn−/− Fcgr2b−/− hFcRn transgenic mice. Because the only version of FcRn that these mice express is human, hIgG stabilization in such mice should occur solely as a consequence of hFcRn. Accordingly, the ability of anti-hFcRn to ameliorate the inflammatory lesions would be evidence that anti-hFcRn blockade provides positive therapeutic benefits in treatment the pathogenic human antibody-induced lesions. Data presented in FIG. 8 shows that DVN24 administration considerably reduced the arthritic lesions compared with administration with the negative control mAb. This exemplary data shows that mAbs directed against a determinant of hFcRn, which the aforementioned studies indicate does not interfere with albumin stabilization, can provide protection against lesions caused by pathological human antibodies. It is therefore envisioned that agents that provide this selective therapeutic blockade of hFcRn's normal protection of hIgG could be used to treat human autoimmune diseases.

### Materials and Methods

Mice. Mice carrying null alleles for FcRn^{tm1Dcr} (Roopenian et al., 2003, J Immunol 170: 3528-33) were backcrossed for a minimum of 10 onto either C57BL/6J (B6) mice. Mice isogenic for human (h) FcRn transgenic (Tg) line hFcRn276, carrying a human FcRn cDNA driven by a heterologous promoter/enhancer were established from independent B6 founder mice, as described (Chaudhury et al., 2003, J Exp Med 197: 315-22; Roopenian et al., 2003, J Immunol 170: 3528-33). Mice deficient for Fcgr2b−/− were obtained from Taconic Farms, Germantown N.Y. FcRn−/− Fcgr2b−/− hFcRn transgenic line 276 mice were produced by intercrossing FcRn−/− hFcRn transgenic line 276 mice with Fcgr2b−/− mice.

In vivo monitoring of tracer human serum albumin (HSA) and hIgG. 100 μg of HSA (biotinylated with N-hydroxysuccinimidobiotin at a 10:1 weight ratio; Sigma-Aldrich, St. Louis, Mo.) and humanized IgG1 (anti-Her-2 IgG₁ kindly provided by G. Meng, Genentech, Inc.) tracers were injected intraperitoneally, as described (Roopenian et al., 2003, J Immunol 170: 3528-33). Blood was serially collected from the retroorbital plexus just before the tracer injection and every 24 hr for 7 days. Anti-Her-2 hIgG₁ antibody tracer in mouse serum was detected by a standard sandwich ELISA, where the capture antigen was Her-2 ligand and the detection antibody was goat anti-hIgG alkaline phosphatase (Southern Biotechnology, Birmingham Ala.). A modified sandwich ELISA protocol was used to detect HSA-biotin in mouse serum, with the diluent buffer substituted by albumin free ELISA wash buffer. Rabbit anti-HSA antibodies (US Biological, 5 μg/ml) was used to capture HAS-biotin and streptavidin alkaline phosphatase (Southern Biotechnology, 1 μg/ml) was used for detection. Clearance was based on the amount of tracer retained relative to that present 24 h after injection.

Generation and validation of hFcRn constructs. hFcRn constructs (CDS, ECTM and ssECTM) were cloned into the pEGFP-C1 vector backbone (BD Biosciences, Franklin Lakes, N.J.). 118 bp of 5′ non-coding sequence and the first 23 amino acids encoding the human FcRn signal sequence were PCR-amplified from human FcRn cDNA (kindly provided by Clark Anderson, Ohio State University) using the following primers: FcRN.SigSeq-F, CCCCCCCCGCTAGCGAAG CCCCTCCTCG GCGTCCTGGT (SEQ ID NO: 2) (NheI site underlined) and FcRN.SigSeq-R, CCCCCCCCACCGGTCCGCCCAGGCTCCCAGG AAGGAGAAA (SEQ ID NO: 3) (AgeI site underlined). Extra bases were included at the 5′ ends of all PCR primers to increase the efficiency of restriction endonuclease activity. This PCR product was inserted downstream of the CMV IE promoter, between the NheI and AgeI restriction sites upstream and in frame with the GFP coding sequence. This intermediate construct was used to produce N-terminal GFP-tagged tail-less hFcRn (ssECTM) described below. In order to produce tail-less FcRn (ssECTM), PCR primers CDS-F and ECTM-R, CCCCCCCCGAATTCttaCCTCATCCTTCTCCACAACAGAGCT (SEQ ID NO: 4) (EcoRI site underlined; premature STOP codon in lower case) were used to amplify codons 24-325 and a premature STOP codon. This PCR product was also inserted into the vector backbone containing the FcRn signal sequence and GFP as described above. Lastly, to generate the non-GFP tagged tail-less FcRn construct, ECTM, the product of PCR primers FcRn.SigSeq-F and ECTM-R was inserted between the NheI and EcoRI sites of the pEGFP-C1 vector resulting in the excision of the GFP coding fragment. All PCR-amplified inserts were bi-directionally sequence verified across the cloning sites.

Cell culture and transfection. Stable HEK293 transfectants were produced similarly using FCS-supplemented DMEM with 800 μg/ml, and then 400 μg/ml G418 (Sigma-Aldrich, St. Louis, Mo.) for selection. The cell lines were then deliberately maintained with a population of hFcRn positive and negative cells for analysis.

Flow cytometry. Confluent adherent HEK293 cells, untransfected or stably expressing GFP-hFcRn (ssECTM) or hFcRn (ECTM) were gently washed once with PBS and harvested after a 5 min incubation at 37° C. with 0.5% trypsin/5.3 mM EDTA in PBS. The activity of trypsin was then blocked by adding DMEM with 5% FCS. Cells were then washed twice in PBS pH 7.4 to remove serum (and albumin), followed by two additional washes with PBS pH 7.4 or pH 6. Human serum albumin (HSA; Sigma-Aldrich, 6 μg/ml), human IgG3 (Calbiochem) Alexafluor₆₄₇ (Molecular Probes, Eugene Oreg.) conjugate (hIgG₃-AF₆₄₇, 100 μg/ml), or 2% human serum in pH 6.0 or 7.4 PBS were used to determine HSA or hIgG binding. These reagents were added to 10⁶ cells in a volume of 50 μl. HSA binding was detected with biotinylated goat anti-HSA antibody (GAHSA-biotin; Antibodies Incorporated, Davis, Calif., 40 μg/ml) or using HSA-biotin. Biotinylated reagents were detected with 2 μg/ml either streptavidin allophycocyanin or streptavidin phycoerythrin (SA-APC or SA-PE; Molecular Probes). hIgG was detected with goat anti-human IgG phycoerythrin (GAH IgG-PE; Southern Biotech, 2 μg/ml). Each reagent was incubated with cells for 1 hr on ice, and the cells were washed between each step to remove unbound reagent. Each incubation step and wash was performed with PBS of the indicated pH. Cells were acquired after propidium iodide exclusion using a FACSCalibur and CellQuest software (Becton-Dickinson, Frankin Lakes, N.J.).

For competition experiments, ssECTM cells were washed 2× at pH 7.5, and serial doses of unlabeled HSA, hIgG (purified from GammaGuard hIgG), or human transferrin (hTfn, Sigma-Aldrich) were preloaded onto 10⁶ ssECTM cells in a volume of 50 μl for 1 hr. Either hIgG-AF₆₄₇ (final concentration 50 μg/ml) or HSA-biotin (final concentration 250 μg/ml) was then added and incubated with ssECTM cells for 60 min. For HSA competition, the cells were then washed two times and stained with SA-PE at 5 μg/ml. After 30 minutes, the cells were washed and flow data of GFP-positive cells were acquired after propidium iodide exclusion. All treatments were performed on ice.

Production and screening of anti-hFcRn mAbs. To produce anti-hFcRn mAbs, B6-FcRn−/− mice were immunized with 2×10⁷ spleen cells from B6 mice transgenic for hFcRn. Sera from the mice were then screened for their ability to specifically bind ssECTM cells. Mice whose serum showed appreciable anti-hFcRn activity were rechallenged with B6-hFcRn transgenic spleen cells and three days later spleen cells from these mice were fused with SP2 for hybridoma production (Cooper and Paterson 2004, supra). Fused cells were cultured for one day in 20%-FBS supplemented DMEM (DMEM20) with 300 U/ml IL6 in flasks to remove adherent fibroblasts, then plated at approximately 2.5×10⁵/100 μl/well into flat bottomed 96 well plates containing 100 μl/well DMEM20 with 2× hypoxanthin/aminopterin/thymidine (HAT) and 300 U/ml IL6. Supernatants from individual wells were screened for specific binding to ssECTM cells in a cellular ELISA. ssECTM cells were plated at 3×10⁵/well into 96 well flat bottomed plates. Supernatants from the 96 well hybridoma cultures were harvested at day 8 to 10 of culture and added to the ssECTM cells after the plates had been centrifuged and decanted. After a 30 minute incubation on ice, cells were washed 2× 300 μl/well with cell ELISA buffer (PBS with 5% FBS and 0.05% NaN₃) by centrifugation and decanting. Goat anti-mouse IgG-alkaline phosphatase (Southern Biotech, Birmingham, Ala.) was diluted 1:1000 in cell ELISA buffer, added at 100 μl/well, and incubated 30 minutes on ice. Cells were washed 2× 300 μl/well with cell ELISA buffer and anti-hFcRn activity was detected using the substrate p-nitrophenyl phosphate (100 μl/well at 1 mg/ml, Sigma, St. Louis, Mo.). Plates were read at an absorbance of 405 nm on an EL212e Microplate Bio-Kinetics Reader (Bio-Tek Instruments, Winooski, Vt.). Hybridoma cells whose supernatants showed absorbance above an optical density (O.D.) of 0.03 were cloned and recloned, and stably growing clones were re-tested in the cellular ELISA. Ascites from selected anti-hFcRn hybridoma clones was then produced in C.B-17-scid mice, purified on HiTrap Protein G columns (Amersham Biosciences, Uppsala, Sweden), and their specificity was confirmed using ssECTM and ECTM cells. Aliquots of each antibody were also labeled with Alexafluor₆₄₇ using a kit (Molecular Probes, Eugene, Oreg.).

Anti-hFcRn mAb blockade of hIgG and HSA binding to hFcRn. To determine how anti-hFcRn mAbs compete with hIgG and HSA for hFcRn binding, serial doses of unlabeled anti-hFcRn mAbs were added to ssECTM cells. After 30 minutes, either hIgG-Alexafluor₆₄₇ (50 μg/ml) or HSA-biotin (250 μg/ml) was added. After 60 minutes, hIgG-Alexafluor₆₄₇ stained cells were washed and acquired. HSA-biotin stained cells were washed two times and stained with SA-PE at 5 μg/ml. After 30 minutes, HSA-biotin stained cells were washed flow cytometric data were acquired. All incubations were on ice and cell washes used 4 ml PBS, pH 6.

Statistical analysis. Statistical analysis was performed by using the non-parametric Rank Sum test or the two tailed Student's T-test. Differences were considered significant when p≦0.05. All values were expressed as mean±standard error of the mean (s.e.m.).

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An isolated antibody or antigen binding portion thereof that binds to an epitope on human FcRn, wherein the antibody or the antigen-binding portion thereof selectively inhibits the binding of the Fc portion of IgG antibody to human FcRn, but does not inhibit the binding of human albumin to human FcRn.

2. The antibody or antigen binding portion thereof of claim 1, wherein the antibody is a monoclonal antibody.

3. The antibody or antigen binding portion thereof of claim 1, wherein the antibody or antigen binding portion thereof can be administered to a human.

4. The isolated antibody or antigen binding portion thereof of claim 1, wherein the antibody or antigen binding portion thereof selectively decreases the serum half-life of a human IgG but does not decrease the serum half-life of human albumin in vivo.

5. The isolated antibody or antigen binding portion thereof of claim 1, wherein the antibody or antigen binding portion thereof ameliorates or inhibits the inflammatory lesions induced by a human autoantibody in a subject.

6. The isolated antibody or antigen binding portion thereof of claim 1, wherein the antibody is selected from the group consisting of antibodies denoted herein as DVN21 and DVN24.

7. The isolated antibody or antigen binding portion thereof of claim 1, wherein the antibody is a recombinant antibody.

8. The isolated antibody or antigen binding portion thereof of claim 1, wherein the antibody is a humanized antibody.

9. The isolated antibody or antigen-binding portion thereof of claim 1, wherein the antibody is a chimeric antibody.

10. The isolated antibody or antigen-binding portion thereof of claim 1, wherein the antibody is a human antibody.

11. The isolated antibody or antigen-binding portion thereof of claim 1, wherein the antibody is a bispecific or multispecific antibody.

12. The isolated antibody or antigen-binding portion thereof of claim 1, wherein the isolated antigen-binding portion is selected from the group consisting of a Fab fragment, a F(ab′)2 fragment, and a Fv fragment CDR3.

13. The isolated antibody or antigen-binding portion thereof of claim 1, wherein the antibody or antigen-binding portion thereof is selected for its ability to bind live cells expressing FcRn.

14. The isolated antibody or antigen-binding portion thereof of claim 1, wherein the FcRn is labeled.

15. The isolated antibody or antigen-binding portion thereof of claim 1, wherein the antibody or antigen-binding portion thereof is selected in vivo for its ability to decrease the serum half-life of a human IgG but does not decrease the serum half-life of human albumin.

16. The isolated antibody or antigen-binding portion thereof of claim 1, wherein the antibody or antigen-binding portion thereof is selected in a transgenic mouse which is deficient in the endogenous FcRn gene but has a transgene encoding human FcRn.

17. The isolated antibody or antigen-binding portion thereof of claim 1, wherein the antibody or antigen-binding portion thereof specifically binds to human FcRn with a binding affinity of at least about 1×10⁻⁸M or less.

18. An isolated antibody or antigen binding portion thereof of claim 1, wherein the isolated antibody or antigen binding portion thereof is covalently linked to an additional functional moiety.

19. The isolated antibody or antigen binding portion thereof of claim 18, wherein the additional functional moiety is a label.

20. The isolated antibody or antigen binding portion thereof of claim 19, wherein the label is a detectable label.

21. The isolated antibody or antigen binding portion thereof of claim 20, wherein the label is selected from the group consisting of a fluorescent label, a radioactive label, and a label having a distinctive nuclear magnetic resonance signature.

22. The isolated antibody or antigen binding portion thereof of claim 18, wherein the additional functional moiety confers increased serum half-life on the antibody or antigen binding portion thereof.

23. The isolated antibody or antigen binding portion thereof of claim 22, wherein the additional functional moiety comprises a polyethylene glycol (PEG) moiety.

24. The isolated antibody or antigen binding portion thereof of claim 22, wherein the additional functional moiety comprises a biotin moiety.

25. A hybridoma cell line that produces an antibody of claim 1.

26. The hybridoma cell line of claim 25, wherein the hybridoma cell line produces an antibody selected from the group consisting of antibodies denoted herein as DVN21 and DVN24.

27. A composition comprising: at least one antibody or antigen-binding portion thereof according to any one of claims 1-24, and a pharmaceutically acceptable carrier, excipient, or stabilizer.

28. The composition of claim 27, further comprising an immunostimulatory agent, an immunomodulator, or a combination thereof.

29. The composition of claim 28, wherein the immunomodulator is selected from alpha-interferon, gamma-interferon, tumor necrosis factor-alpha or a combination thereof.

30. The composition of claim 28, wherein the immunostimulatory agent is selected from interleukin-2, immunostimulatory oligonucleotides, or a combination thereof.

31. An isolated nucleic acid molecule encoding an isolated antibody or antigen-binding portion thereof of any one of claims 1-24.

32. A method for inhibiting FcRn mediated IgG protection in an individual, comprising administering the antibody of claim 1 to an individual in need thereof in sufficient amounts to selectively inhibit binding of human FcRn to a human IgG but not to human albumin.

33. The method of claim 32, wherein the individual has an autoimmune disease.

34. The method of claim 32, wherein the individual has systemic lupus erythematosus.

35. A method of preventing or treating an autoimmune disease in a patient, comprising administering the antibody of claim 1 to a patient in sufficient amounts to prevent or treat the autoimmune disease.

36. The method of claim 35, wherein the autoimmune disease is selected from the group consisting of systemic lupus erythematosus, insulin resistant diabetes, myasthenia gravis, polyarteritis, autoimmune thrombocytopenic purpura, cutaneous vasculitis, bullous pemphigoid, pemphigus vulgaris, pemphigus foliaceus, Goodpasture's syndrome, rheumatoid arthritis, Kawasaki's disease, and Sjogren's syndrome.

37. The method of claim 35, wherein the isolated antibody or antigen binding portion thereof is administered systemically.

38. The method of claim 35, wherein the isolated antibody is administered locally.

39. The method of claim 35, further comprising administering to a patient an immunomodulator.

40. An in vitro method of identifying an inhibitor that selectively inhibits binding of human FcRn to a human IgG but not to human albumin, comprising:
a) contacting a candidate inhibitor with human FcRn and a human IgG under conditions appropriate for binding of the human FcRn to the human IgG;
b) assaying for binding of human FcRn to the human IgG in the presence of the candidate inhibitor, as compared to binding of human FcRn to the human IgG in the absence of candidate inhibitor;
c) contacting a candidate inhibitor to human FcRn and human albumin under conditions appropriate for binding of the human FcRn to human albumin; and
d) assaying for binding of human FcRn to human albumin in the presence of the candidate inhibitor, as compared to binding of human FcRn to human albumin in the absence of candidate inhibitor,
wherein if the candidate inhibitor inhibits binding of human FcRn to the human IgG but not to human albumin, the candidate inhibitor is an inhibitor that selectively inhibits binding of human FcRn to a human IgG but not to human albumin.

41. An in vitro method of identifying an inhibitor that selectively inhibits binding of human FcRn to a human IgG but not to human albumin, comprising:
a) contacting a candidate inhibitor with human FcRn, a human IgG, and human albumin;
b) assaying for binding of human FcRn to the human IgG in the presence of the candidate inhibitor, as compared to binding of human FcRn to the human IgG in the absence of candidate inhibitor; and
c) assaying for binding of human FcRn to human albumin in the presence of the candidate inhibitor, as compared to binding of human FcRn to human albumin in the absence of candidate inhibitor,
wherein if the candidate inhibitor inhibits binding of human FcRn to the human IgG but not to human albumin, the candidate inhibitor is an inhibitor that selectively inhibits binding of human FcRn to a human IgG but not to human albumin.

42. An in vivo method of identifying an agent that selectively reduces the half-life of human IgG but not the half-life of human albumin, comprising:
a) administering a candidate agent and a tracer human IgG to an FcRn^{-/-}/huFcRn⁺ transgenic mouse;
b) determining the half-life of the tracer human IgG in the mouse in the presence of the candidate agent, as compared to the half-life of the tracer human IgG in the absence of candidate agent;
c) administering the candidate agent and a tracer human albumin to the FcRn^{-/-}/huFcRn⁺ transgenic mouse; and
d) determining the half-life of the tracer human albumin in the mouse in the presence of the candidate agent, as compared to the half-life of the tracer human albumin in the absence of candidate agent,
wherein if the candidate agent reduces the half-life of the tracer human IgG but not the half-life of the tracer human albumin, the candidate agent is an agent that selectively reduces the half-life of human IgG but not the half-life of human albumin.

43. An in vivo method of identifying an agent that selectively reduces the half-life of human IgG but not the half-life of human albumin, comprising:
a) administering a candidate agent, a tracer human IgG, and a tracer human albumin to an FcRn^{-/-}/huFcRn⁺ transgenic mouse;
b) determining the half-life of the tracer human IgG in the mouse in the presence of the candidate agent, as compared to the half-life of the tracer human IgG in the absence of candidate agent; and
c) determining the half-life of the tracer human albumin in the mouse in the presence of the candidate agent, as compared to the half-life of the tracer human albumin in the absence of candidate agent,
wherein if the candidate agent reduces the half-life of the tracer human IgG but not the half-life of the tracer human albumin, the candidate agent is an agent that selectively reduces the half-life of human IgG but not the half-life of human albumin.

44. The method of any of claims 40-43, wherein the agent is selected from the group consisting of an antibody, a polypeptide, a synthetic peptide, a peptidomimetic, and a small molecule.

45. The method of claim 44, wherein the agent is a fusion protein comprising an Fc portion of an IgG polypeptide.

46. The method of claim 44, wherein the agent is an Fc portion of an IgG polypeptide.

47. Use of an isolated antibody or antigen binding portion thereof of claim 1 to make a pharmaceutical preparation for treating an autoimmune disease.

48. The use of claim 47, wherein the antibody is a monoclonal antibody.

49. Use of an isolated antibody or antigen binding portion thereof of claim 1 to promote clearance of radioactive antibodies or antibody conjugated toxins used for imaging or treatment of cancer.

50. The use of claim 49, wherein the antibody is a monoclonal antibody.
